(19) United States
(12) Patent Application Publication
(10) Pub. No.: US 2008/0319533 A1
(43) Pub. Date: Dec. 25, 2008

(54) ANEURYSM OCCLUSION ASSIST DEVICE

(75) Inventor: Cathleen von Lehe, Rogers, MN (US)

Correspondence Address: SCHWEGMAN, LUNDBERG & WOESSNER, P.A., P.O. BOX 2938, MINNEAPOLIS, MN, 55402, US

(73) Assignee: NEUROVASX, INC., N. MAPLE GROVE, MN (US)

(21) Appl. No.: 11/767,130

(22) Filed: Jun. 22, 2007

(51) Int. Cl. A61F 2/06

(52) U.S. Cl. 623/1.22

(57) ABSTRACT

Embodiments of the invention described herein include an aneurysm occlusion assist device comprising a main body effective for providing support to a blood vessel, defining an open volume sized to permit substantially unimpeded blood flow when the main body is implanted in a bifurcated aneurysm.

### FIELD

The inventive subject matter described herein relates to an aneurysm occlusion assist device embodiments and to method embodiments for making the aneurysm occlusion assist device and method repairing an aneurysm.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the products, processes and data as described below and in the tables that form a part of this document: Copyright 2007, Neurovasx, Inc. All Rights Reserved.

### BACKGROUND OF THE INVENTION

An aneurysm is a balloon-like swelling in a wall of a blood vessel. Aneurysms result in weakness of the vessel wall in which it occurs. This weakness predisposes the vessel to tear or rupture with potentially catastrophic consequences for any individual having the aneurysm. Vascular aneurysms are a result of an abnormal dilation of a blood vessel, usually resulting from disease and/or genetic predisposition which can weaken the arterial wall and allow it to expand. Aneurysm sites tend to be areas of mechanical stress concentration so that fluid flow seems to be the most likely initiating cause for the formation of these aneurysms.

Aneurysms in cerebral circulation tend to occur in an anterior communicating artery, posterior communicating artery, and a middle cerebral artery. The majority of these aneurysms arise from either curvature in the vessels or at bifurcations of these vessels. The majority of cerebral aneurysms occur in women. Cerebral aneurysms are most often diagnosed by the rupture and subarachnoid bleeding of the aneurysm.

Cerebral aneurysms are most commonly treated in open surgical procedures where the diseased vessel segment is clipped across the base of the aneurysm. While considered to be an effective surgical technique, particularly considering an alternative which may be a ruptured or re-bleed of a cerebral aneurysm, conventional neurosurgery suffers from a number of disadvantages. The surgical procedure is complex and requires experienced surgeons and well-equipped surgical facilities. Surgical cerebral aneurysm repair has a relatively high mortality and morbidity rate of about 2% to 10%.

Current treatment options for cerebral aneurysm fall into two categories, surgical and interventional. The surgical option has been the long held standard of care for the treatment of aneurysms. Surgical treatment involves a long, delicate operative procedure that has a significant risk and a long period of postoperative rehabilitation and critical care. Successful surgery allows for an endothelial cell to endothelial cell closure of the aneurysm and therefore a cure for the disease. If an aneurysm is present within an artery in the brain and bursts, this creates a subarachnoid hemorrhage, and a possibility that death may occur. Additionally, even with successful surgery, recovery takes several weeks and often requires a lengthy hospital stay.

A presentation of a technique referred to as the “Sacred Technique” at an ASNR poster exhibit P129 at the Univ. of Iowa demonstrated a technique in which a stent was placed into an aneurysm to provide a base structure to assist in coil placement. This technique was specific for bifurcated aneurysms shown at 10 in prior art FIG. 1 as the stent 12 entered the aneurysm in an up-down configuration rather than a side configuration as shown in prior art FIG. 1.

In order to overcome some of these drawbacks, interventional methods and prostheses have been developed to provide an artificial structural support to the vessel region impacted by the aneurysm. The structural support must have an ability to maintain its integrity under blood pressure conditions and impact pressure within an aneurysmal sac and thus prevent or minimize a chance of rupture. U.S. Pat. No. 5,405,379 to Lane, discloses a self-expanding cylindrical tube which is intended to span an aneurysm and result in isolating the aneurysm from blood flow. While this type of stent-like device may reduce the risk of aneurysm rupture, the device does not promote healing within the aneurysm. Furthermore, the stent may increase a risk of thrombosis and embolism. Additionally, the wall thickness of the stent may undesirably reduce the fluid flow rate in a blood vessel. Stents typically are not used to treat aneurysms in a bend in an artery or in tortuous vessels such as in the brain because stents tend to straighten the vessel.

U.S. Pat. No. 5,354,295 to Guglielmi et al., describes a type of vasoclusion coil. Disadvantages of use of this type of coil are that the coil may compact, may migrate over time, and the coil does not optimize the patient's natural healing processes.

### IN THE FIGURES

FIG. 1 is a cross-sectional view of a prior art assist device for coil placement.

FIG. 2 is a cross-sectional view of an assist device embodiment of the invention.

FIG. 3 is a cross-sectional view of the assist device embodiment of FIG. 2 shown positioned in situ.

FIG. 4 is a cross-sectional view of another assist device embodiment of the invention.

FIG. 5 is a cross-sectional view of the assist device embodiment of FIG. 4 shown positioned in situ.

FIG. 6 is a cross-sectional view of another assist device embodiment of the invention.

FIG. 7 is a cross-sectional view of the assist device embodiment of FIG. 6 shown positioned in situ.

FIG. 8 is a cross-sectional view of another assist device embodiment of the invention.

FIG. 9 is a cross-sectional view of the assist device embodiment of FIG. 8 shown positioned in situ.

FIG. 10 is a cross-sectional view of another assist device embodiment that includes a microcatheter.

FIGS. 11a and 11b are a cross-sectional views of the assist device embodiment of FIG. 10 in situ.

FIG. 12 is a cross-sectional in situ view of the assist device embodiment of FIG. 10 that also includes an embolic material.

FIG. 13 is a cross-sectional view of one construction embodiment of the assist device embodiment of FIG. 10.

FIG. 14 is a cross-sectional view of another construction embodiment of the assist device embodiment of FIG. 10.

### DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the aneurysm filler detacher wire embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition levels are in reference to the active level of that component or composition, and are exclusive of impurities, for example, residual solvents or by-products, which may be present in commercially available sources.

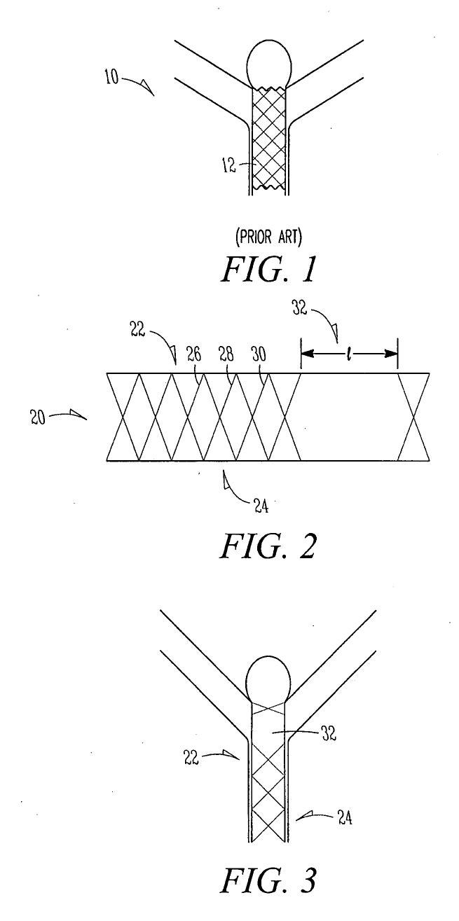

Embodiments described herein include base structure embodiments for assisting in coil placement into an aneurysm occlusion that minimize impediments to blood flow, such as the struts shown in the prior art structure of FIG. 1. One embodiment is shown at 20 in FIG. 2. The structure of FIG. 2 includes extension legs 22 and 24 and support components 26, 28, 30, for example, that contact each of the extension legs 22 and 24. The structure 20 defines an open volume 32 that corresponds to the volume between two branches of a blood vessel, as shown at 30 in FIG. 3. The structure 20 can be constructed to define a variety of volumes to accommodate a variety of sizes of side branches.

The extension legs 22 and 24 are a single, fine wire obstacle rather than multiple struts as is shown in the prior art structure of FIG. 1. For some embodiments, the extension legs 22 and 24 and, optionally, the support components 26, 28, and 30 are made of round wire to eliminate sharp edges and to reduce thrombus formation.

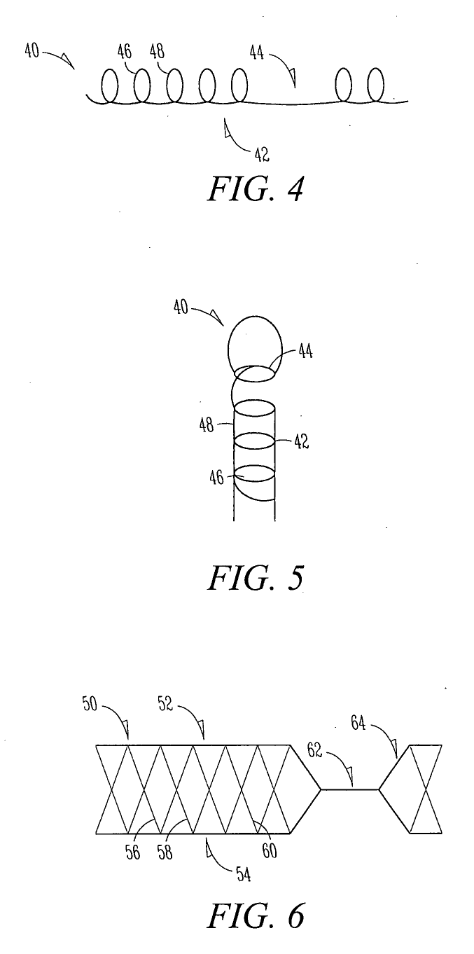

Another base structure embodiment is shown at 40 in FIGS. 4 and 5. The base structure embodiment includes a main body coil 42 that defines a plurality of subcoils, such as 46, and 48 and a space 44 that is sized to minimize blood flow restriction in a blood vessel branch as shown in FIG. 5. The structure 20 can be constructed to define a variety of volumes to accommodate a variety of sizes of side branches. For some embodiments, the coil 44 is made of round wire to eliminate sharp edges and to reduce thrombus formation.

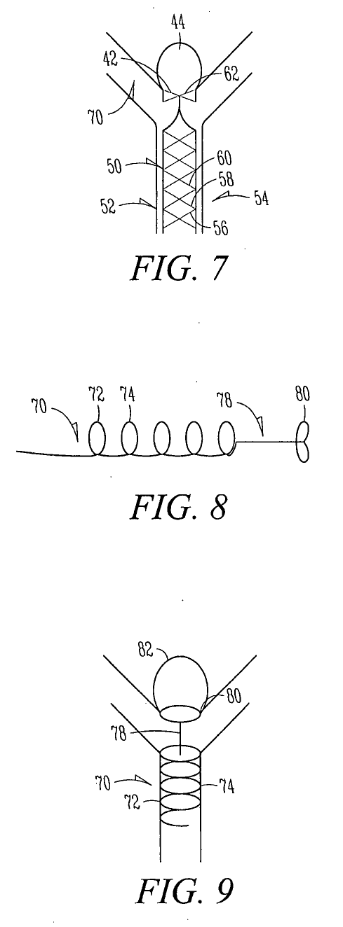

One other base structure embodiment, shown at 50 in FIGS. 6 and 7, includes extension legs 52 and 54 and support components 56, 58, 60, for example, that contact each of the extension legs 52 and 54. The structure 50 defines an open volume 62 that corresponds to the volume between two branches of a blood vessel, as shown at 70 in FIG. 7. The structure 50 can be constructed to define a variety of volumes to accommodate a variety of sizes of side branches.

The extension legs 52 and 54 are a single, fine wire obstacle rather than multiple struts as is shown in the prior art structure of FIG. 1. For some embodiments, the extension legs 52 and 54 and, optionally, the support components 56, 58, and 60 are made of round wire to eliminate sharp edges and to reduce thrombus formation.

The structure 50 also includes an adjacent structural component 64 that acts to provide support adjacent the walls of an aneurysm 66.

Another embodiment is shown at 70 in FIGS. 8 and 9. The base structure embodiment 70 includes a main body coil 72 that defines a plurality of subcoils, such as 72, and 74 and a space 74 that is sized to minimize blood flow restriction in a blood vessel branch as shown in FIG. 5. The structure 70 can be constructed to define a variety of volumes to accommodate a variety of sizes of side branches. For some embodiments, the coil 72 is made of round wire to eliminate sharp edges and to reduce thrombus formation.

The base structure embodiment 70 also includes a structure 80 that acts to provide support adjacent the walls of an aneurysm 86.

All of the embodiments shown herein provide a scaffold or base to the neck of an aneurysm to allow the aneurysm to be filled with the embolic coils or other embolic agents and reduce the likeliness that the coils and other base embodiments will back out of the aneurysm during fill. All of the base embodiments described herein are implantable. Some embodiments may be biodegradable.

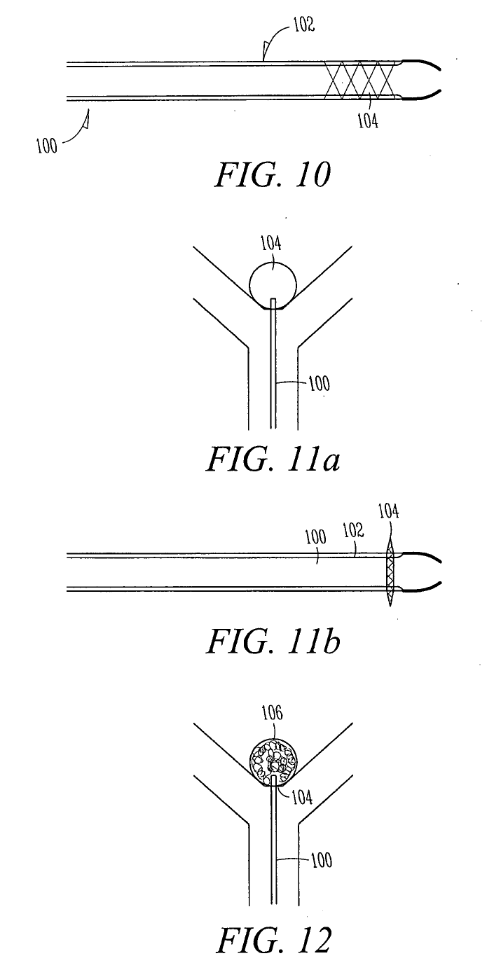

Another aneurysm occlusion assist device is shown at 100 in FIG. 10. The embodiment 100 includes a microcatheter 102 and a base structure 104 built into the microcatheter 102. The microcatheter 102 that includes the base structure 104 has a flat conformation for transport and insertion into an aneurysm. Once positioned within the aneurysm, the microcatheter 100 is activated to an expanded conformation, as shown in FIG. 11. In the expanded conformation, the embodiment 100 covers the base of the aneurysm and allows embolic coils 106 or other devices to pass through the microcatheter and fill the aneurysm, as shown in FIG. 12. Once the aneurysm is packed with embolic material, the element 100 can be deactivated from an expanded conformation and can be removed, leaving only the embolic material as implant material.

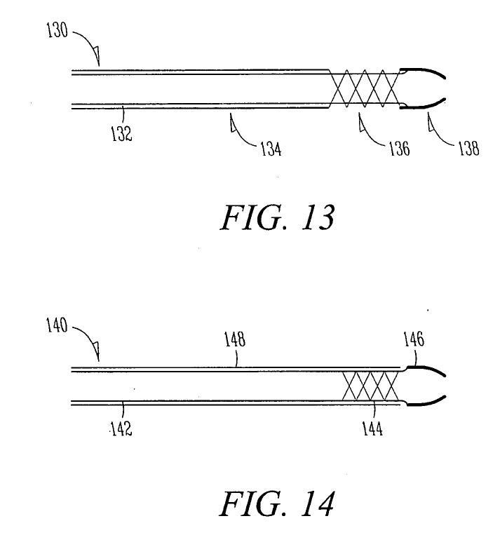

One image illustrating construction of the embodiment 100 is shown in FIG. 13, at 130. The image 13 shows an inner shaft 132 and an outer shaft 134 that are not connected to each other but can move back and forth relative to one another. The outer shaft 134 is connected to a braid 136 and the braid 136 is connected to a tip 138. The inner shaft 132 is connected only to the tip 138. With this embodiment, the outer shaft 134 can be pushed forward relative to the inner shaft 132, compressing the braid 136, allowing the braid 136 to expand outward. The braid 136 is, for some embodiments, nitinol and may have shape memory that enhances its ability to expand and take the shape of the neck of an aneurysm.

Another construction embodiment is shown at 140 in FIG. 14. For this construction, a braid 144 is attached to an inner shaft 142 and tip 146. An outer shaft 148 covers the braid 144 during advancement to an aneurysm and during positioning within the aneurysm. Once positioned, the outer shaft 148 can be moved backwards, exposing the braid 144, allowing the braid 144 to take a pre-set shape.

For some embodiments, lubricious materials such as hydrophilic materials may be used to coat the base structure embodiments. One or more bioactive materials may also be included in the composition of the core. The term “bioactive” refers to any agent that exhibits effects in vivo, for example, a thrombotic agent, a therapeutic agent, and the like. Examples of bioactive materials include cytokines; extra-cellular matrix molecules (e.g., collagen); trace metals (e.g., copper); matrix metalloproteinase inhibitors; and other molecules that stabilize thrombus formation or inhibit clot lysis (e.g., proteins or functional fragments of proteins, including but not limited to Factor XIII, C2-antiplasmin, plasminogen activator inhibitor-1 (PAI-1) or the like)). Examples of cytokines that may be used alone or in combination in practicing the invention described herein include basic fibroblast growth factor (bFGF), platelet derived growth factor (pDGF), vascular endothelial growth factor (VEGF), transforming growth factor beta (TGF-β), and the like. Cytokines, extra-cellular matrix molecules, and thrombus stabilizing molecules are commercially available from several vendors such as Genzyme (Framingham, Mass.), Genentech (South San Francisco, Calif.), Amgen (Thousand Oaks, Calif.), R&D Systems, and Immunex (Seattle, Wash.). Additionally, bioactive polypeptides can be synthesized recombinantly as the sequence of many of these molecules are also available, for example, from the GenBank database. Thus, it is intended that embodiments of the invention include use of DNA or RNA encoding any of the bioactive molecules.

Furthermore, molecules having similar biological activity as wild-type or purified cytokines, matrix metalloproteinase inhibitors, extra-cellular matrix molecules, thrombus-stabilizing proteins such as recombinantly produced or mutants thereof, and nucleic acid encoding these molecules may also be used. The amount and concentration of the bioactive materials that may be included in the composition of the core member may vary, depending on the specific application, and can be determined by one skilled in the art. It will be understood that any combination of materials, concentration, or dosage can be used so long as it is not harmful to the subject.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

What is claimed is:

1. An aneurysm occlusion assist device comprising a main body effective for providing support to a blood vessel, defining an open volume sized to permit substantially unimpeded blood flow when the main body is implanted in a bifurcated aneurysm.

2. The aneurysm occlusion assist device of claim 1 wherein the main body comprises a braid configuration.

3. The aneurysm occlusion assist device of claim 1 wherein the main body comprises a coil configuration.

4. The aneurysm occlusion assist device of claim 1, further comprising a main body component positioned adjacent to the main body, the main body component effective for providing structural support to the aneurysm.

5. The aneurysm occlusion assist device of claim 1, further comprising a microcatheter, wherein the main body is built into the microcatheter.
